(19) United States
(12) Patent Application Publication
(10) Pub. No.: US 2021/0307347 A1
(43) Pub. Date: Oct. 7, 2021

(54) METHOD FOR OBTAINING POLYLACTIC ACID (PLA) FROM CHEESE WHEY

(71) Applicant: Laura Viviana Cuervo Garces, Bogota (CO)

(72) Inventor: Laura Viviana Cuervo Garces, Bogota (CO)

(21) Appl. No.: 17/262,767

(22) PCT Filed: Apr. 8, 2019
(86) PCT No.: PCT/IB2019/052879

§ 371 Date: Jan. 25, 2021

(30) Foreign Application Priority Data
Jul. 26, 2018 (CO) NC2018/0007766

(51) Int. Cl. A23C 21/02; C08G 63/85; C08G 63/08; C08G 63/90; C12P 7/56

(52) U.S. Cl. CPC A23C 21/026; C08G 63/85; C12P 7/56; C08G 63/90; C08G 63/08

(57) ABSTRACT

The application discloses a method for obtaining polylactic acid (PLA) from cheese whey, which comprises, inter alia, steps of deproteinisation and inoculation, fermentation, separation, polymerisation and drying. The claimed method includes the use of microorganisms such as Lactobacillus delbrueckii and Streptococcus thermophilus. In addition, the separation step includes an adsorption process in a tower packed with activated cationic zeolite, which exchanges H⁺ for Ca⁺, and the solvent n-pentane is used in said separation step to extract lactic acid, with recovery of 90% of the lactic acid obtained by fermentation.

### TECHNICAL FIELD

The present invention is related to a method of obtaining polylactic acid (PLA) from cheese whey.

### BACKGROUND OF THE INVENTION

Currently, the most significant residue of the cheese industry is the whey, where approximately 80% of the milk that enters the process ends up being discarded as whey, which is a residue that contaminates 10 times more than what is allowed. The whey has several useful properties, but due to the lack of technologies and/or studies it is not used in Colombia. On the other hand, the industry of the biopolymers in the country is very scarce and unknown, at national level there is no producer of polylactic acid (PLA)—coming from the whey—which is an “environmentally friendly” polymer with great utilities in the plastic or pharmaceutical industry. In addition, new local policies have been established in Colombia for the non-use of oil-based bags and the use of recyclable or biodegradable bags is being encouraged, motivating various sectors to import PLA for its production.

On the other hand, through the decrees proposed by the National Institute of Surveillance of Drugs and Foods (INVIMA—Regulatory Entity in Colombia) that regulate dumping and environmental problems, it is decreed that the serum is a waste that contaminates 10 times more than what is allowed. Large cheese companies hire other companies to make the final disposal of the waste, small companies on the other hand dilute the serum and dump it, this leads to fines and sanctions by the regulatory body.

Research publications have demonstrated the use of lactic acid, as well as the polymeric form of lactic acid, the polylactic acid used for composting. Also, it is found that polylactic acid has application as a biodegradable plastic in the food industry, cosmetics and medicine.

Lactic and polylactic acid and their traditional production have been described in different documents, for example the application with reference number RU2016112880 and entitled “METHOD OF PRODUCING LACTIC ACID AND POLYLACTIC ACID” reveals a method to produce lactic acid that includes filtering an aqueous solution containing lactic acid through a nanofiltration membrane to recover an aqueous solution of lactic acid from the permeate side (Stage A); distilling the aqueous lactic acid solution to recover lactic acid from the vapor side (Stage B); and crystallizing the lactic acid obtained in Stage B, and performing solid-liquid separation to recover a lactic acid crystal(s) (Stage C). In spite of the above, this process does not reveal all the conditions set forth in our application with the advantages and results that we will explain later.

### SUMMARY OF THE INVENTION

The problem stated in this application, is the need for a method to reuse a waste that is highly polluting to water sources known as whey.

To solve this problem, the present invention provides a process of transformation of the above-mentioned residue into a polymeric material called polylactic acid (PLA), which presents biodegradable, thermally resistant and thermally stable characteristics guaranteeing the care of the environment, by collection of the whey and the production of a biopolymer that can be used in the plastic industry. It is expected that the product will give an added value to the whey, this comes from the production of cheese and is considered a by-product.

An important aspect of the invention is that the obtained polylactic acid is a material that besides its varied characteristics possesses different applications, such as the elaboration of bags, packaging, bottles, straws, fabric fibers, 3D printing and knee prosthesis. This material solves another environmental problem, the accumulation of plastics in waste deposits and water sources.

The method of obtaining the material consists of five stages, which are: Deproteinization and inoculation, fermentation (obtaining lactic acid), separation and purification of lactic acid, polymerization of lactic acid and drying of polylactic acid.

### BRIEF DESCRIPTION OF THE DRAWINGS

To complement this description and in order to help a better understanding of the characteristics of the invention, according to the preferred examples of the practical embodiments, as an integral part of this description, a group of drawings is accompanied where, for illustrative and not restrictive purposes, the following has been represented:

FIG. 1 shows a Ternary diagram of the components in the separation.

FIG. 2 shows the reaction mechanisms of polylactic acid.

FIG. 3A shows a process flow diagram where an output of the microfilter is received by the calcium lactate and water shown in FIG. 3B.

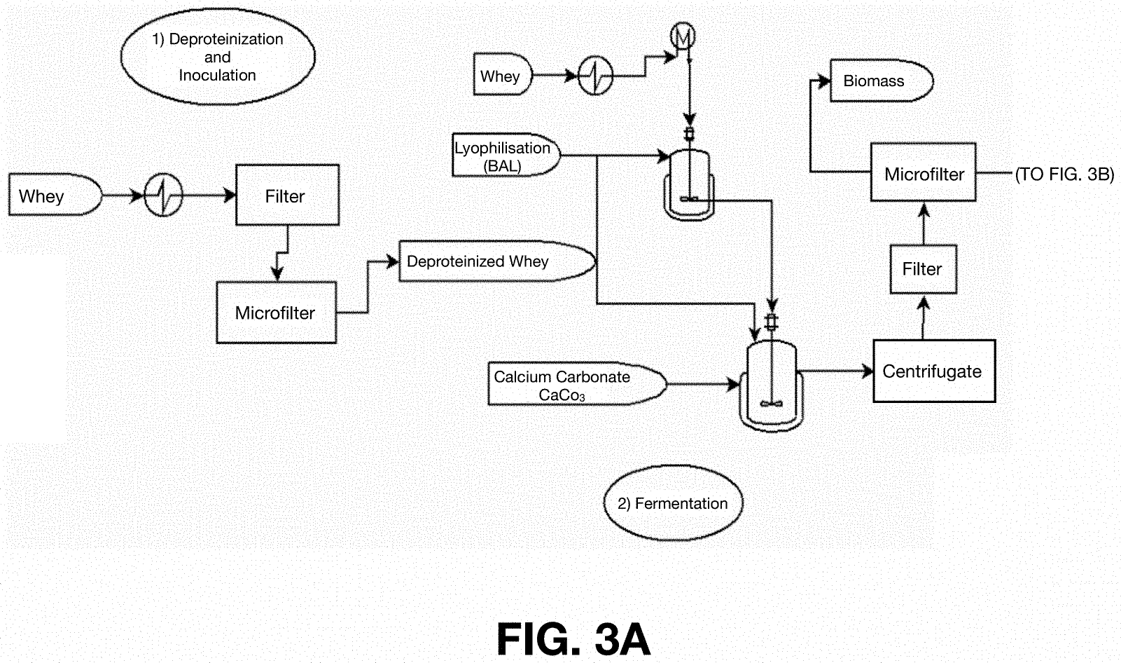

FIG. 3B shows a process flow diagram receiving an output of the microfilter shown in FIG. 3A, where an output of the mix of Lactic acid and Sulfuric acid (60%) is received by the Lactide shown in FIG. 3C.

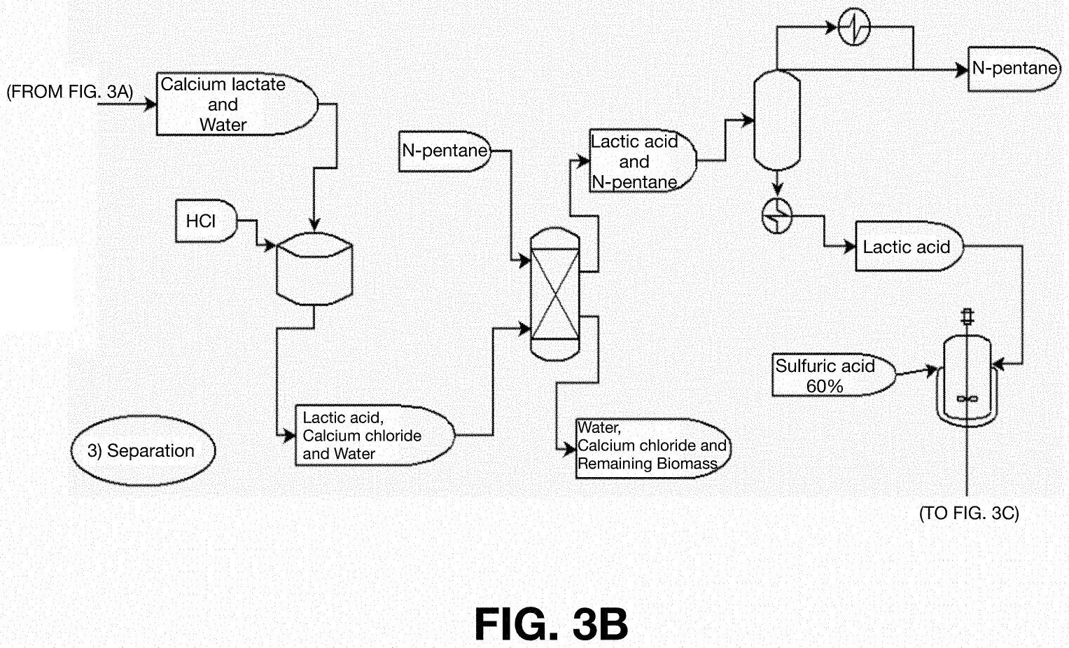

FIG. 3C shows a process flow diagram receiving the mix of Lactic acid and Sulfuric acid (60%) shown in FIG. 3B.

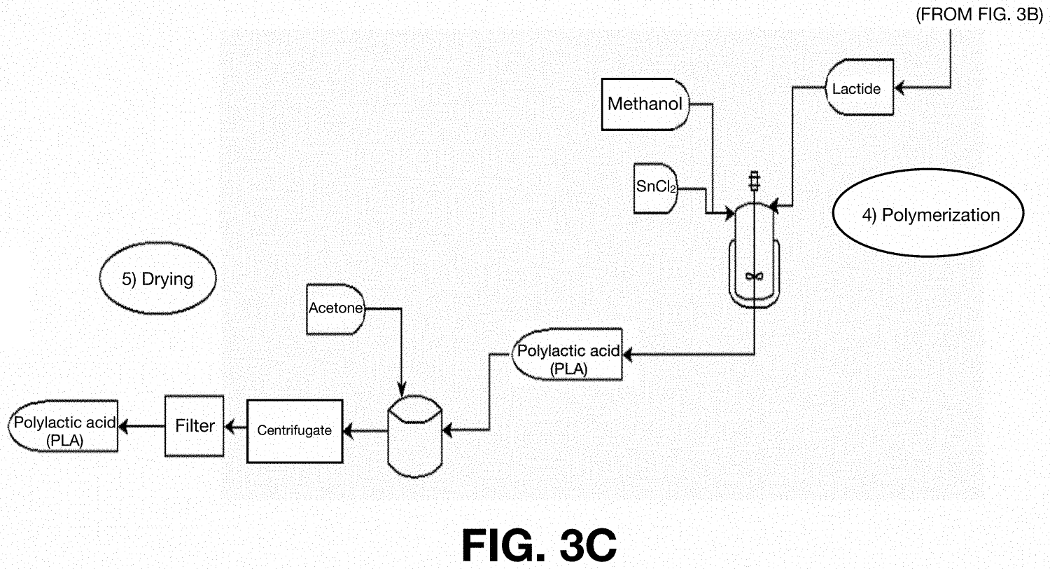

### DETAILED DESCRIPTION OF THE INVENTION

The present invention develops a biodegradable polymer called polylactic acid from cheese whey. Basically, the problem that the product under study solves is environmental pollution both from the dumping of the cheese whey as a final disposal and from the accumulation of conventional plastics in landfills and garbage dumps. The polylactic acid is a biodegradable polymer, it degrades in less than 1 year under composting, next, the common physical characteristics that the material possesses.

In accordance with the above, the obtention of polylactic acid and polycaprolactone (PLA copolymer) is presented, where a method of hydroxypropylation of the cassava starch is performed so that the fermentation process can be carried out.

TABLE 1
Polylactic acid physical properties

| Formula | (C₃H₄O₂)ₙ |
|---|---|
| Molecular weight (Kda) | 50-750 |
| Melting point (° C.) | 178 |
| Density (g/cm3) | 1.25 |
| % Crystallinity | 15-74 |
| Tensile strength (Mpa) | 40-60 |
| Tension module (Gpa) | 3-4 |
| Max. temperature used (° C.) | 50-125 |
| Tg glass transition temperature | 60-65 |

Source: L. Sema C and collaborators Et. Al. Year 2003

Next, the method of obtaining the material from the cheese whey, with its contributing chemical characteristics, as raw material in the process of obtaining, is presented.

The milk is a colloid that contains different components like proteins, lactose, fat and water, next the chemical composition of the milk is shown in table 2.

TABLE 2
Milk composition

| Main component | Variation limits | Mean value |
|---|---|---|
| Water | 85.5-89.5 | 87.5 |
| Total of solids | 10.5-14.5 | 13.0 |
| Fat | 2.5-6.0 | 3.9 |
| Proteins | 2.9-5.0 | 3.4 |
| Lactose | 3.6-5.5 | 4.8 |
| Minerals | 0.6-0.9 | 0.8 |

Source: Manual from the Tetrapak dairy industry

Only 20% of the milk is used in the cheese industry, where the cheese is made from casein, by precipitation either acid or an enzymatic agent called rennet or chymosin. The rest of the milk is whey, the chemical composition is presented in the table, it contains a high content of nutrients and above all lactose, which makes it ideal for a fermentation process.

TABLE 3
Whey composition

| Component | Whey % | Casein whey |
|---|---|---|
| Total of solids | 6.4 | 6.5 |
| Water | 93.6 | 93.5 |
| Fat | 0.05 | 0.04 |
| Protein | 0.55 | 0.55 |
| NNP | 0.18 | 0.18 |
| Lactose | 4.8 | 4.9 |
| Ashes (Mineral salts) | 0.5 | 0.8 |
| Calcium | 0.043 | 12 |
| Phosphorus | 0.040 | 0.065 |
| Sodium | 0.050 | 0.050 |
| Potassium | 0.16 | 0.16 |
| Chloride | 0.11 | 0.11 |
| Lactic acid | 0.05 | 0.4 |

Source: Manual from the Tetrapak dairy industry

As previously mentioned, whey is a waste for the dairy industry and its final disposal is as a dumping for water sources, the decree 3930 of 2010 and resolution 635 of 2015 stipulate that the allowed for industrial liquid waste in the indexes of COD (Chemical Oxygen Demand) and BOD (Biological Oxygen Demand) is between 3000 and 4000, the whey contains between 30,000 and 40,000 in indexes of COD and BOD.

Therefore, a study was carried out at laboratory level in which and a preliminary study of a scaling process, where certain chemical processes were carried out to the whey obtained from the cheese production to achieve the referenced polymer.

Method of Obtaining Polylactic Acid from Cheese Whey

Now a basic engineering process for the production of polylactic acid from whey is proposed, it should be noted that this waste has never been used as a raw material for the production of polylactic acid, the stages of the process are:

1) Deproteinization and inoculation: Whey is a residue in the cheese industry that has high contents of lactose, proteins and water which are remnants of cheese production. The whey from the cheese producing companies is collected and due to the different studies carried out, it was discovered that it was better in the process to deproteinize the whey by denaturing the proteins with a thermal shock at temperatures above 100° C., then it is filtered and microfiltered with the objective of concentrating the whey with the help of a vacuum pump. The microorganisms used are freeze-dried lactic acid bacteria, where an inoculation that lasts 24 hours at the conditions of 38° C. to 42° C., pH between 5 and 6 must be performed, depending on the volume that is going to be fermented the agitation varies, the agitation used in lactic processes of yogurt preparation is taken as a reference, the inoculation is made in a solution of 80% deproteinized whey and 20% non-deproteinized whey (pasteurized), which is 10% in total volume that is going to be carried out of fermentation.

2) Fermentation: The fermentation process is carried out adding the inoculum after 24 hours and the total volume is completed with deproteinized whey, the process is anaerobic and the same conditions are used from 38° C. to 42° C., pH between 5 and 6, depending on the volume that is going to be fermented the agitation varies, to carry out an adequate control of pH, calcium carbonate is added. Fermentation lasts approximately 72 to 96 hours and is carried out in order to obtain lactic acid. Therefore, the bacteria to be used must be homofermentative and therefore must be lactic acid microorganisms as: Lactobacillus delbrueckii and Streptococcus thermophilus.

It is important to note that by fermentation a racemic mixture of lactic acid enantiomers D (−) and L (+) is produced. Lactic acid production was evaluated by IR spectrophotometry and titratable acid test with NTC 4978 standard. The IR spectrophotometer test showed only the existence of L (+) isomers of lactic acid. Fermentation was carried out in a 2.5 L capacity bioreactor, with a RUSHTON type agitator using a 12V motor, where temperature control was automated and carried out by means of an Arduino microcontroller and heat exchange, controlling a heating resistor by means of a relay.

The whey was supplemented with 15% excess lactose and 5% yeast extract, which after 72 hours was shown by titratable acidity to yield 13.02 g/L of lactic acid in the form of calcium lactate. A higher quantity than the one obtained from the prior art disclosed in the degree work “Evaluation of the Polylactic Acid Synthesis from the Cheese Factory Whey”.

3) Separation: Biomass, lactic acid in the form of calcium lactate and water are produced when the fermentation is finished; therefore, it is necessary to remove the biomass through a centrifugation process at 5000 RPM and a vacuum filtration and microfiltration is performed with membranes having a pore size of 1.2 μm.

Afterwards, the pH is reduced by 2 or 3 with hydrochloric acid (HCl) in the calcium lactate and water solution. To produce lactic acid, calcium chloride and water, an adsorption is performed on activated carbon and anionic zeolite to capture the calcium ions present in the solution. Lactic acid and water are related compounds, that is why a thorough study on the separation and recovery of lactic acid has been carried out and the evaluation was continued with more solvents.

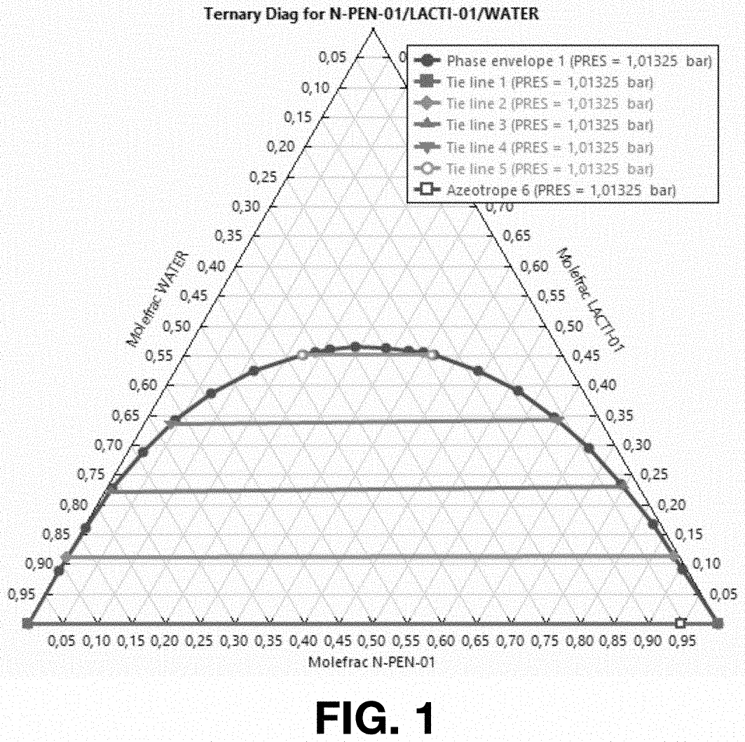

Surprisingly, the solvent that yielded the best results was n-pentane because it recovers 90% of the lactic acid produced during fermentation, where the calculated by titratable acidity was 11.71 g/L. It should also be noted that this is the first time that n-pentane is used to separate lactic acid and it showed an improvement with respect to what was obtained with the prior art study for separation with diethyl ether proposed by the author J. Cuellar (Year 2014) and L. Cuervo and J. Echeverry (Year 2016). The process used is a liquid-liquid extraction adding in a proportion of 50% v/v of n-pentane solvent to the water solution and an agitation is made, so that there is more contact between the phases, it is waited until two phases are evidenced and they are separated in different containers. The lactic acid is separated from the water by dragging the solvent and the calcium chloride obtained by reducing the pH remains in the aqueous phase. Then, by means of a single stage distillation, the solvent is recovered, using the boiling point of pentane; it is important to indicate that pentane is a totally insoluble solvent in water, which facilitates the interaction of lactic acid with the solvent and this can be removed from the aqueous solution. FIG. 1 shows the ternary diagram used for the separation with the n-pentane solvent, made with the Aspen Properties software.

4) Polymerization: The ORP (Open Ring Opening) method is used in the polymerization process> It was found in the prior art that the ideal percentage for the polymerization of lactic acid is 88 to 92% of purity, the lactic acid obtained was ideal for polymerization after separated and purified. The first step is the elaboration of a pre-polymer with lactic acid in a vacuum catalytic reaction with an acid compound, this pre-polymer is called lactide. The second step is a catalytic reaction of tin with the pre-polymer and tin chloride II (SnCl₂) is used as a catalyst. In addition, methanol is used as a reaction initiator.

As previously mentioned, the polylactic acid is obtained by a polymerization of the lactic acid by the open ring method (ORP), where an esterification with the obtained lactic acid is performed. This process is carried out by a reaction with sulphuric acid of 50% to 80% v/v of purity at a heating temperature of 120° C. to 150° C. and constant agitation for approximately 8 hours, with constant additions of sulfuric acid. Once the lactic acid diester called lactide is obtained, the open ring polymerization reaction (ORP) is carried out. This consists of a catalytic reaction where the lactide obtained is added, methanol and tin chloride II are added. This reaction occurs between 1 and 4 hours with the generation of a white powder which refers to the polylactic acid.

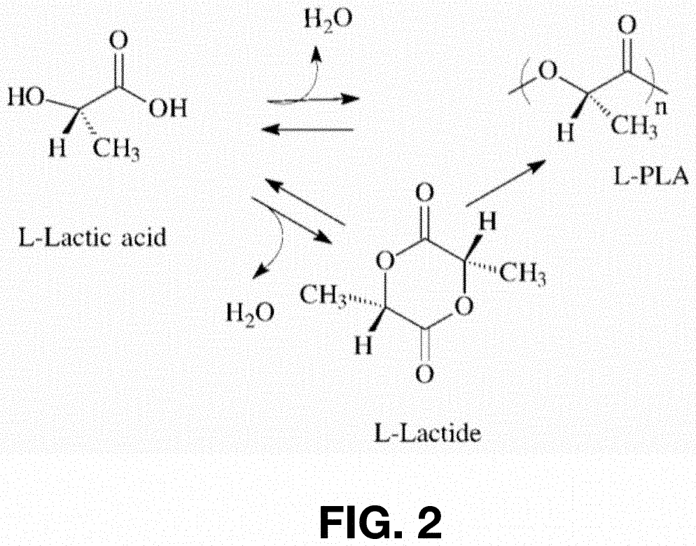

Polylactic acid is obtained by two methods of polymerization, which are direct polycondensation and the ROP method. The advantages of performing the ROP method, is that a high molecular weight polymer is generated, this process is determined by the method of polymerization index and X-ray diffraction test, this is compared with the prior art and authors such as J. Cortés (Year 2016) and P. Pagesa (Year 2010). It was found that the polymer presents crystallinity characteristics and when carrying out the test by X-ray diffraction and comparing it with what was obtained by the previously mentioned authors demonstrated the characteristic peak where the intensity is measured and the angle at which it presents this property, this angle is between 15 and 20°, the PLA obtained by the present invention is at 18°. The reaction mechanism by the two existing polymerization methods is shown in FIG. 2 (Source: Lactic acid polymerization Extracted biopolymers article extracted from: http://www.eis.uva.es/˜biopolimeros/monica/Polimerizacion.htm).

5) Drying: When the polymerization is finished and the ethanol is evaporated, acetone is added to the powder to remove impurities and it is taken to a centrifuge at 5000 rpm, then a vacuum filtration is carried out and the powder is taken to a dryer to obtain the completely dry PLA.

FIG. 2 shows a flow chart of the process from obtaining the whey to the polymer (Source: The authors DIAW software).

Advantages of the inventive process:

1) The fermentative process was carried out in a fermenter, and the substrate was conditioned to obtain a percentage of yield of 34%, using the microorganisms (Lactobacillus Delbrueckii and Streptococcus Thermophilus).

2) The separation process includes an adsorption process in a tower packed with activated cationic zeolite, which performs an exchange of H⁺ for Ca⁺. N-pentane is used as a solvent in the extraction of lactic acid, a solvent that due to its insolubility and low boiling point characteristics has improved the process with respect to the prior art. Since 90% of the lactic acid obtained by fermentation was recovered.

3) The polymerization process, when carried out by the ORP method, with tin chloride as catalyst represents a 99% yield with respect to the lactic acid obtained in the fermentation, this material has demonstrated to comply with physical properties that commercial PLA possesses, it is soluble in chloroform, it has a boiling point of 121° C., it is crystalline and it has the characteristic peak in X-ray diffraction tests. The advantage of ORP polymerization over the direct polycondensation method is not only in time, energy expenditure but in the production of a high molecular weight polymer. Unlike conventional processes where the direct polycondensation method is used to obtain polylactic acid, which has more stages and produces a low molecular weight polymer.

4) The main advantage of the process, as it comes from the cheese whey, is that it proposes to use a highly contaminating waste and generate a substitute for plastic materials, mitigating both contamination of river sources by dumping the waste, as well as the accumulation of conventional plastics. The material decomposes and degrades in less than 1 year under compost.

1-6. (canceled)

7. A method of obtaining polylactic acid (PLA) from cheese whey, said method comprising the steps of:
deproteinizing whey by denaturing proteins of said whey with thermal shock at temperatures above 100° C. and concentrating said whey by filtering and micro-filtering said whey;
preparing an inoculum by agitating a mixture of freeze-dried lactic acid bacteria and a solution of deproteinized whey and pasteurized whey at a temperature between 38° C. to 42° C., and a pH between 5 and 6;
fermenting a mixture of calcium carbonate, said inoculum and said deproteinized whey, wherein said calcium carbonate is added to achieve a pH between 5 and 6 and the fermentation process is carried out at a temperature between 38° C. to 42° C.;
centrifugating, filtering and micro-filtering said fermented mixture to separate biomass from a solution of calcium lactate and water;
adding hydrochloric acid (HCl) to the solution of calcium lactate and water, to produce lactic acid, calcium chloride and water at a pH between 2 or 3;
adsorbing said solution of lactic acid, calcium chloride and water in activated carbon and anionic zeolite to capture calcium ions present in the solution;
adding n-pentane to said solution in a proportion of 50% v/v to separate lactic acid from the water by liquid-liquid extraction forming a first phase containing n-pentane and lactic acid and a second phase containing the calcium chloride and water;
distilling said first phase in a single stage to recover said n-pentane and lactic acid;
preparing from said recovered lactic acid a lactide pre-polymer with an acid compound and performing a catalytic reaction of a tin-based compound with said lactide pre-polymer;
reacting said lactide pre-polymer with sulfuric acid from 50% to 80% v/v of purity to esterify said lactide pre-polymer at a temperature from 120° C. to 150° C. in order to obtain polylactic acid (PLA); and
drying said obtained polylactic acid (PLA) to obtain completely dry polylactic acid (PLA).

8. The method of obtaining polylactic acid (PLA) according to claim 1, wherein the fermentation stage is carried out in a fermenter with a substrate conditioned to obtain a yield of 34%.

9. The method of obtaining polylactic acid (PLA) according to claim 1, wherein said freeze-dried lactic acid bacteria comprises homofermentative microorganisms Lactobacillus Delbrueckii and Streptococcus Thermophilus.

10. The method of obtaining polylactic acid (PLA) according to claim 1, wherein said calcium ions present in the solution are captured by exchange of H⁺ for Ca⁺ during the adsorption step.

11. The method of obtaining polylactic acid (PLA) according to claim 1, wherein 90% of the calcium lactate obtained by the fermentation step is recovered at the separation step.

12. The method of obtaining polylactic acid (PLA) according to claim 1, wherein said lactide pre-polymer is esterified by an open ring polymerization (ORP) method.

13. The method of obtaining polylactic acid (PLA) according to claim 1, wherein said inoculated mixture comprises 10% of a total volume to be fermented.

14. The method of obtaining polylactic acid (PLA) according to claim 1, further comprising adding excess lactose and yeast extract for the fermentation step.

15. The method of obtaining polylactic acid (PLA) according to claim 1, wherein said catalytic reaction is carried out for 1 to 4 hours.

16. The method of obtaining polylactic acid (PLA) according to claim 1, wherein said solution of deproteinized whey and pasteurized whey comprises 80% deproteinizing whey and 20% pasteurized whey.

17. The method of obtaining polylactic acid (PLA) according to claim 1, wherein tin-based compound is tin chloride II (SnCl₂).
